(19) United States
(12) Patent Application Publication
(10) Pub. No.: US 2023/0114739 A1
(43) Pub. Date: Apr. 13, 2023

(54) STRUCTURAL BRAIN BIOMARKERS OF SUICIDE RISK IN ADOLESCENTS

(71) Applicant: Father Flanagan's Boys' Home Doing Business as Boys Town National Research Hospital, Omaha, NE (US)

(72) Inventor: Sahil Bajaj, Boys Town, NE (US)

(21) Appl. No.: 17/938,538

(22) Filed: Oct. 6, 2022

Related U.S. Application Data
(60) Provisional application No. 63/262,271, filed on Oct. 8, 2021.

(51) Int. Cl. G16H 50/20; G06N 20/10

(52) U.S. Cl. CPC G16H 50/20; G06N 20/10

(57) ABSTRACT

A machine learned model has been trained using structural brain data obtained from parcellated brain images. The brain data is used to train the model to identify classifiers associated with biomarkers of specific regions of the brain. The biomarkers indicate the presence and/or potential for one or more mental conditions, such as the potential for suicide risk. Using the trained model, clinics, hospitals, and other locations will be able to quickly and accurately determine the potential for mental conditions, which can aid in identifying treatment for patients. The results can provide a prognosis for the mental condition as well.

### CROSS REFERENCE TO RELATED APPLICATIONS

This application claims priority under 35 U.S.C. § 119 to provisional patent application U.S. Ser. No. 63/262,271, filed Oct. 8, 2021. The provisional patent application is herein incorporated by reference in its entirety, including without limitation, the specification, claims, and abstract, as well as any figures, tables, appendices, or drawings thereof.

### STATEMENT REGARDING FEDERALLY SPONSORED RESEARCH OR DEVELOPMENT

This invention was made with government support under Grant award number K22-MH109558 awarded by the National Institute of Mental Health. The government has certain rights in the invention.

### FIELD OF THE INVENTION

The invention relates generally to systems and/or methods for identifying structures of the brain. More particularly, but not exclusively, the invention relates towards systems and/or methods for analyzing brain structures to determine, diagnose, and/or treat potential mental and/or behavioral disorders.

### BACKGROUND OF THE INVENTION

Mental and behavioral disorders include behavioral or mental patterns that causes significant distress or impairment of personal functioning. Many disorders have been described, with signs and symptoms that vary widely between specific disorders. The causes of mental disorders are often unclear. Theories may incorporate findings from a range of fields. Mental disorders are usually defined by a combination of how a person behaves, feels, perceives, or thinks. This may be associated with functions and structures of particular regions of the brain, often in a social context. A mental disorder is one aspect of mental health.

Suicide is one of the leading causes of death for individuals aged 10-19 years in the United States. There has been a 76% increase in suicide rate in 15-19 years age group between 2007 and 2017. Increased risk of suicide is seen in a variety of clinical conditions including major depressive disorder (MDD), anxiety disorders, and conduct disorders. The emerging statistics and the association between suicide risk and a variety of clinical conditions indicate that its very crucial to improve our understanding about neural signatures of suicide risk.

Prior work has been able to show accuracy of 78.6%, sensitivity (i.e., ability to designate an individual with disease as positive) of about 73.2% and specificity (i.e., ability to designate an individual without disease as negative) of about 84% to distinguish suicide attempters and patients with suicidal ideation but without attempts. However, prior work is lacking the identification of patients at suicide risk from typically developing individuals by implementing whole-brain sophisticated machine-learning approaches in adolescents using multiple classification algorithms.

Previous work has focused on the neurobiology of suicide risk via a variety of neuroimaging modalities, including the resting-state and task-based functional MRI (fMRI), diffusion-weighted MRI, and structural MRI (sMRI). Most of the sMRI studies have shown widespread structural alterations in the dorsal and ventral prefrontal cortices, inferior frontal cortex, and middle and superior temporal cortices.

However, no previous work has ever utilized one of the finest whole-brain parcellations to date (i.e., 1000-area parcellation), in conjunction with a cutting-edge two-step, two-layer cross-validation (also called Nested Cross-Validation [CV]) machine-learning approach.

Thus, there exists a need in the art for systems and associated methods for reviewing whole-brain parcellations to determine a nexus between structural markers in the brain and associated mental and/or behavioral disorders or other issues.

### SUMMARY OF THE INVENTION

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the invention to improve on or overcome the deficiencies in the art.

It is a further object, feature, and/or advantage address the previous challenges and use cutting-edge machine learning approach on structural MRI (sMRI) data to develop reliable structural biomarkers of suicide risk with reliable accuracy, sensitivity, specificity, and area under the receiver operating characteristic curve (AUC) parameters.

It is yet another object, feature, and/or advantage to determine structural biomarkers associated with one or more mental and/or behavioral disorders. The biomarkers can be used to identify, diagnose, determine prognosis, and/or treat the associated mental and/or behavioral disorders.

It is still yet a further object, feature, and/or advantage to examine cortical volume [CVol] across 1000 cortical regions and sub-cortical volume (SCV) across 12 sub-cortical regions in adolescents at suicidal risk relative to typically developing (TD) adolescents and further determine a classification model for CVol/SCV that best classifies adolescents at suicidal risk from TD adolescents.

It is still a further object, feature, and/or advantage to utilize machine learning and training of the same to quickly and accurately diagnose potential mental and/or behavioral disorders or other issues at early stages.

The systems and/or methods disclosed herein can be used in a wide variety of applications. For example, it is envisioned that any of the systems and/or methods could be used in clinics, hospitals, schools, other medical facilities, or the like, wherein it may be beneficial to identify potential mental and/or behavioral issues and to determine the prognosis of the same at an early stage.

The system has added importance in being accurate and effective in determining said potential mental and/or behavioral issues to allow for diagnosis, prognosis, and/or treatment to be applied at said early stage in order to provide the best possible outcome for a patient.

According to some aspects of the present disclosure, a system for diagnosing mental issues comprises at least one processor and at least one memory configured to implement a deployed learning network model. The deployed network model is generated from a training network, wherein the training network trained with a method comprising the steps of: reviewing a plurality of brain images comprising a plurality of regions of the brain; and identifying a classifier of the brain images that corresponds with one or more mental issues. The deployed learning network is stored on one or more non-transitory computer readable media comprising instructions comprising: comparing the deployed learning network model with brain images of a patient to determine if the brain images of the patient correspond to the one or more mental issues associated with the trained classifier.

According to at least some aspects and/or embodiments, the network model comprises a nested cross-validation approach.

According to at least some aspects and/or embodiments, the training network comprises a. a support vector machine (SVM) method, b. a k-Nearest Neighbors (k-NN) method, or c. an ensemble trees (ENS) method.

According to at least some aspects and/or embodiments, the training network method further comprises fining an estimated and generalized accuracy of the deployed network model.

According to at least some aspects and/or embodiments, the brain images comprise whole-brain parcellation into 1000-cortical regions and 12-subcortical regions.

According to at least some aspects and/or embodiments, the 1000-cortical regions are separated into 500 regions per hemisphere.

According to at least some aspects and/or embodiments, the 12-subcortical regions are separated into 6 regions per hemisphere.

According to at least some aspects and/or embodiments, the brain images are adjusted with respect to age, sex, IQ, and head size.

According to at least some aspects and/or embodiments, the one or more mental issues comprises risk of suicide.

According to additional aspects, a computer implemented method comprises training a network via a processor, wherein the training the network comprises: reviewing a plurality of parcellated brain images; identifying one or more classifiers of the parcellated brain images that corresponds to one or more mental conditions; and storing the classifiers in a memory associated with the processor; using the trained network to compare stored classifiers with brain images of a patient to diagnose one or more mental conditions of the patient.

According to at least some aspects and/or embodiments, the method further comprises determining a treatment for the diagnosed mental condition based upon the diagnosis with the trained network.

According to at least some aspects and/or embodiments, the one or more mental conditions comprises suicide risk.

According to at least some aspects and/or embodiments, the plurality of parcellated brain images comprise images separated into 1000-cortical regions and 12-subcortical regions.

According to at least some aspects and/or embodiments, the plurality of parcellated brain images comprise structural MRI (sMRI) data.

According to at least some aspects and/or embodiments, the one or more classifiers were determined using a support vector machine learning model.

According to at least some aspects and/or embodiments, the one or more identified classifiers comprise biomarkers associated with brain regions.

According to still further aspects and/or embodiments disclosed, a method for identifying mental issues comprises training a network stored on a processor with memory by reviewing a plurality of brain images; identifying one or more classifiers of the brain images that corresponds to a mental condition; and storing the classifiers in the memory; using the trained network to compare stored classifiers with brain images of a patient to identify potential of the mental condition of the patient.

According to at least some aspects and/or embodiments, the plurality of brain images comprises parcellated brain images separated into 1000-cortical regions and 12-subcortical regions.

According to at least some aspects and/or embodiments, the training network comprises a. a support vector machine (SVM) method, b. a k-Nearest Neighbors (k-NN) method, or c. a classification ensemble (ENS) method.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Several embodiments in which the invention can be practiced are illustrated and described in detail, wherein like reference characters represent like components throughout the several views. The drawings are presented for exemplary purposes and may not be to scale unless otherwise indicated.

FIG. 1A is a depiction of whole-brain parcellation into 1000-cortical regions, including 500 regions per hemisphere.

FIG. 1B is a depiction of whole-brain sub-cortical parcellation into 12-subcortical brain regions, including six regions per hemisphere.

FIG. 2 is an illustration of an exemplary machine learning algorithm according to aspects and/or embodiments of the present disclosure.

FIG. 3A is a depiction showing identification of bilateral outer left and right brain features/regions of interest corresponding to CVol/SCV according to aspects and/or embodiments of the present disclosure.

FIG. 3B is an inner depiction of FIG. 3A.

FIG. 3C is a depiction of right and left bilateral features from a forward perception.

FIG. 3D is a depiction of right and left bilateral features from a rearward direction.

FIG. 3E is a depiction of right and left bilateral features from a bottom perception.

An artisan of ordinary skill need not view, within isolated figure(s), the near infinite number of distinct permutations of features described in the following detailed description to facilitate an understanding of the invention.

### DETAILED DESCRIPTION OF THE INVENTION

The present disclosure is not to be limited to that described herein. Mechanical, electrical, chemical, procedural, and/or other changes can be made without departing from the spirit and scope of the invention. No features shown or described are essential to permit basic operation of the invention unless otherwise indicated.

Unless defined otherwise, all technical and scientific terms used above have the same meaning as commonly understood by one of ordinary skill in the art to which embodiments of the invention pertain.

The terms “a,” “an,” and “the” include both singular and plural referents.

The term “or” is synonymous with “and/or” and means any one member or combination of members of a particular list.

The terms “invention” or “present invention” are not intended to refer to any single embodiment of the particular invention but encompass all possible embodiments as described in the specification and the claims.

The term “about” as used herein refer to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the invention is defined by the appended claims, along with the full scope of equivalents to which such claims are entitled. The scope of the invention is further qualified as including any possible modification to any of the aspects and/or embodiments disclosed herein which would result in other embodiments, combinations, subcombinations, or the like that would be obvious to those skilled in the art.

Aspects and/or embodiments of the present disclosure aim to overcome and/or improve on issues and challenges raised. At least one goal is to use cutting-edge machine learning on structural MRI (sMRI) data to develop reliable structural biomarkers of suicide risk and other mental conditions with reliable accuracy, sensitivity, specificity, and AUC parameters.

Aspects and/or embodiments of the present disclosure utilize whole-brain parcellations (e.g., 1000-area parcellations in some exemplary discussions), in conjunction with a cutting-edge two-step, two-layer cross-validation (also called Nested Cross-Validation [CV]) machine-learning approach. Aspects and/or examples provided utilize these techniques to examine cortical volume [CVol] across 1000 cortical regions and sub-cortical volume (SCV) across 12 sub-cortical regions in adolescents at suicidal risk relative to typically developing (TD) adolescents and further determine a classification model for CVol/SCV that best classifies adolescents at suicidal risk from TD adolescents.

As will be understood, aspects and/or embodiments disclosed herein will utilize processors, memory, instructions, and the like, and will include a machine learning model or models to identify classifiers of aspects of the brain image data (i.e., the parcellated brain images). The classifiers will be used to identify regions or other biomarkers of the brain image that is associated with one or more potential mental conditions. While aspects of the present disclosure may refer to the mental condition of suicide or suicide risk, it should be appreciated that any of the feature disclosed herein could be used for other types of mental conditions, including, but not limited to, depression, aggression, anxiety, or conduct disorders. The classifiers identified by the machine learning model provided can be used for any number of applications and the model can be trained to identify biomarkers associated with generally any mental condition.

Machine learning (ML) is the study of computer algorithms that can improve automatically through experience and by the use of data. It is seen as a part of artificial intelligence. Machine learning algorithms build a model based on sample data, known as “training data”, in order to make predictions or decisions without being explicitly programmed to do so.

While it is envisioned that generally any type of ML (e.g., supervised learning, unsupervised learning, semi-supervised learning, or reinforcement learning) can be utilized by any of the aspects and/or embodiments of the present disclosure utilize supervised learning. Supervised learning (SL) is the machine learning task of learning a function that maps an input to an output based on example input-output pairs. It infers a function from labeled training data consisting of a set of training examples. In supervised learning, each example is a pair consisting of an input object (typically a vector) and a desired output value (also called the supervisory signal). A supervised learning algorithm analyzes the training data and produces an inferred function, which can be used for mapping new examples. An optimal scenario will allow for the algorithm to correctly determine the class labels for unseen instances. This requires the learning algorithm to generalize from the training data to unseen situations in a “reasonable” way (see inductive bias). This statistical quality of an algorithm is measured through the so-called generalization error.

To solve a given problem of supervised learning, one has to perform the following steps: (1) Determine the type of training examples. Before doing anything else, the user should decide what kind of data is to be used as a training set. In the case of handwriting analysis, for example, this might be a single handwritten character, an entire handwritten word, an entire sentence of handwriting or perhaps a full paragraph of handwriting. (2) Gather a training set. The training set needs to be representative of the real-world use of the function. Thus, a set of input objects is gathered, and corresponding outputs are also gathered, either from human experts or from measurements. (3) Determine the input feature representation of the learned function. The accuracy of the learned function depends strongly on how the input object is represented. Typically, the input object is transformed into a feature vector, which contains a number of features that are descriptive of the object. The number of features should not be too large, because of the curse of dimensionality; but should contain enough information to accurately predict the output. (4) Determine the structure of the learned function and corresponding learning algorithm. For example, the engineer may choose to use support-vector machines or decision trees. (5) Complete the design. Run the learning algorithm on the gathered training set. Some supervised learning algorithms require the user to determine certain control parameters. These parameters may be adjusted by optimizing performance on a subset (called a validation set) of the training set, or via cross-validation. (6) Evaluate the accuracy of the learned function. After parameter adjustment and learning, the performance of the resulting function should be measured on a test set that is separate from the training set.

As will be understood, while generally any type of SL can be utilized, the example provided herein utilized three different classification algorithms to train the model, namely the support vector machine (SVM), k-Nearest Neighbors (k-NN), and classification ensemble (ENS).

Support-vector machines (SVMs, also support-vector networks) are supervised learning models with associated learning algorithms that analyze data for classification and regression analysis. Given a set of training examples, each marked as belonging to one of two categories, an SVM training algorithm builds a model that assigns new examples to one category or the other, making it a non-probabilistic binary linear classifier (although methods such as Platt scaling exist to use SVM in a probabilistic classification setting). SVM maps training examples to points in space so as to maximize the width of the gap between the two categories. New examples are then mapped into that same space and predicted to belong to a category based on which side of the gap they fall.

The k-nearest neighbors algorithm (k-NN) is a non-parametric classification method. k-NN is a type of classification where the function is only approximated locally, and all computation is deferred until function evaluation. Since this algorithm relies on distance for classification, if the features represent different physical units or come in vastly different scales then normalizing the training data can improve its accuracy dramatically.

Classification ensemble may also be referred to as ensemble learning. Ensemble methods use multiple learning algorithms to obtain better predictive performance than could be obtained from any of the constituent learning algorithms alone. Unlike a statistical ensemble in statistical mechanics, which is usually infinite, a machine learning ensemble consists of only a concrete finite set of alternative models, but typically allows for much more flexible structure to exist among those alternatives.

As will be understood, the SVM has been shown to provide the best results for the training of the trained model for the brain image data provided.

The trained model and the associated machine learning and application of the model will utilize processors, modules, memories, databases, networks, and potentially user interfaces to show the results and allow changes to be made.

In communications and computing, a computer readable medium is a medium capable of storing data in a format readable by a mechanical device. The term “non-transitory” is used herein to refer to computer readable media (“CRM”) that store data for short periods or in the presence of power such as a memory device.

One or more embodiments described herein can be implemented using programmatic modules, engines, or components. A programmatic module, engine, or component can include a program, a sub-routine, a portion of a program, or a software component or a hardware component capable of performing one or more stated tasks or functions. A module or component can exist on a hardware component independently of other modules or components. Alternatively, a module or component can be a shared element or process of other modules, programs, or machines.

The system will preferably include an intelligent control (i.e., a controller) and components for establishing communications. Examples of such a controller may be processing units alone or other subcomponents of computing devices. The controller can also include other components and can be implemented partially or entirely on a semiconductor (e.g., a field-programmable gate array (“FPGA”)) chip, such as a chip developed through a register transfer level (“RTL”) design process.

A processing unit, also called a processor, is an electronic circuit which performs operations on some external data source, usually memory or some other data stream. Non-limiting examples of processors include a microprocessor, a microcontroller, an arithmetic logic unit (“ALU”), and most notably, a central processing unit (“CPU”). A CPU, also called a central processor or main processor, is the electronic circuitry within a computer that carries out the instructions of a computer program by performing the basic arithmetic, logic, controlling, and input/output (“I/O”) operations specified by the instructions. Processing units are common in tablets, telephones, handheld devices, laptops, user displays, smart devices (TV, speaker, watch, etc.), and other computing devices.

The memory includes, in some embodiments, a program storage area and/or data storage area. The memory can comprise read-only memory (“ROM”, an example of non-volatile memory, meaning it does not lose data when it is not connected to a power source) or random-access memory (“RAM”, an example of volatile memory, meaning it will lose its data when not connected to a power source). Examples of volatile memory include static RAM (“SRAM”), dynamic RAM (“DRAM”), synchronous DRAM (“SDRAM”), etc. Examples of non-volatile memory include electrically erasable programmable read only memory (“EEPROM”), flash memory, hard disks, SD cards, etc. In some embodiments, the processing unit, such as a processor, a microprocessor, or a microcontroller, is connected to the memory and executes software instructions that are capable of being stored in a RAM of the memory (e.g., during execution), a ROM of the memory (e.g., on a generally permanent basis), or another non-transitory computer readable medium such as another memory or a disc.

Generally, the non-transitory computer readable medium operates under control of an operating system stored in the memory. The non-transitory computer readable medium implements a compiler which allows a software application written in a programming language such as COBOL, C++, FORTRAN, or any other known programming language to be translated into code readable by the central processing unit. After completion, the central processing unit accesses and manipulates data stored in the memory of the non-transitory computer readable medium using the relationships and logic dictated by the software application and generated using the compiler.

In one embodiment, the software application and the compiler are tangibly embodied in the computer-readable medium. When the instructions are read and executed by the non-transitory computer readable medium, the non-transitory computer readable medium performs the steps necessary to implement and/or use the present invention. A software application, operating instructions, and/or firmware (semi-permanent software programmed into read-only memory) may also be tangibly embodied in the memory and/or data communication devices, thereby making the software application a product or article of manufacture according to the present invention.

The database is a structured set of data typically held in a computer. The database, as well as data and information contained therein, need not reside in a single physical or electronic location. For example, the database may reside, at least in part, on a local storage device, in an external hard drive, on a database server connected to a network, on a cloud-based storage system, in a distributed ledger (such as those commonly used with blockchain technology), or the like.

It is envisioned that the machine learned model and any of the training of the same could include cloud computing. Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service.

A cloud computing environment is service oriented with a focus on statelessness, low coupling, modularity, and semantic interoperability. At the heart of cloud computing is an infrastructure comprising a network of interconnected nodes.

As noted, the training model could be implemented on a user interface. The interface could also be a point on introduction of data, such as training data or test data to compare to the trained model for analysis. The results of the comparison could then be shown on a user interface.

A user interface is how the user interacts with a machine. The user interface can be a digital interface, a command-line interface, a graphical user interface (“GUI”), oral interface, virtual reality interface, or any other way a user can interact with a machine (user-machine interface). For example, the user interface (“UI”) can include a combination of digital and analog input and/or output devices or any other type of UI input/output device required to achieve a desired level of control and monitoring for a device. Examples of input and/or output devices include computer mice, keyboards, touchscreens, knobs, dials, switches, buttons, speakers, microphones, LIDAR, RADAR, etc. Input(s) received from the UI can then be sent to a microcontroller to control operational aspects of a device.

The user interface module can include a display, which can act as an input and/or output device. More particularly, the display can be a liquid crystal display (“LCD”), a light-emitting diode (“LED”) display, an organic LED (“OLED”) display, an electroluminescent display (“ELD”), a surface-conduction electron emitter display (“SED”), a field-emission display (“FED”), a thin-film transistor (“TFT”) LCD, a bistable cholesteric reflective display (i.e., e-paper), etc. The user interface also can be configured with a microcontroller to display conditions or data associated with the main device in real-time or substantially real-time.

Any components of the system could be connected via network or other communication protocol to transfer information, communicate with other systems, or provide other connectivity. In some embodiments, the network is, by way of example only, a wide area network (“WAN”) such as a TCP/IP based network or a cellular network, a local area network (“LAN”), a neighborhood area network (“NAN”), a home area network (“HAN”), or a personal area network (“PAN”) employing any of a variety of communication protocols, such as Wi-Fi, Bluetooth, ZigBee, near field communication (“NFC”), etc., although other types of networks are possible and are contemplated herein. The network typically allows communication between the communications module and the central location during moments of low-quality connections. Communications through the network can be protected using one or more encryption techniques, such as those techniques provided by the Advanced Encryption Standard (AES), which superseded the Data Encryption Standard (DES), the IEEE 802.1 standard for port-based network security, pre-shared key, Extensible Authentication Protocol (“EAP”), Wired Equivalent Privacy (“WEP”), Temporal Key Integrity Protocol (“TKIP”), Wi-Fi Protected Access (“WPA”), and the like.

### EXAMPLE

The following is an example to aid in explaining and enabling aspects and/or embodiments of the present disclosure. The example should not be seen as limiting the disclosure, or any claims herein, and should be seen as exemplary to aid in the understanding of said disclosure. It should be appreciated that many changes, alternatives, combinations, omissions, or the like, may be made to the following, while still being with the aspects and/or embodiments of the present disclosure.

Data Collection. The example includes sMRI data collected from 158 youths [79 adolescents with clinically concerning levels of suicide risk (mean age=16.26±1.18 years, 43 males) and 79 demographically matched typically developing [TD] adolescents (mean age=15.94±1.48 years, 53 males)] between 13 and 19 years of age. The Suicide Probability Scale (SPS) was used to determine the severity of suicide risk.

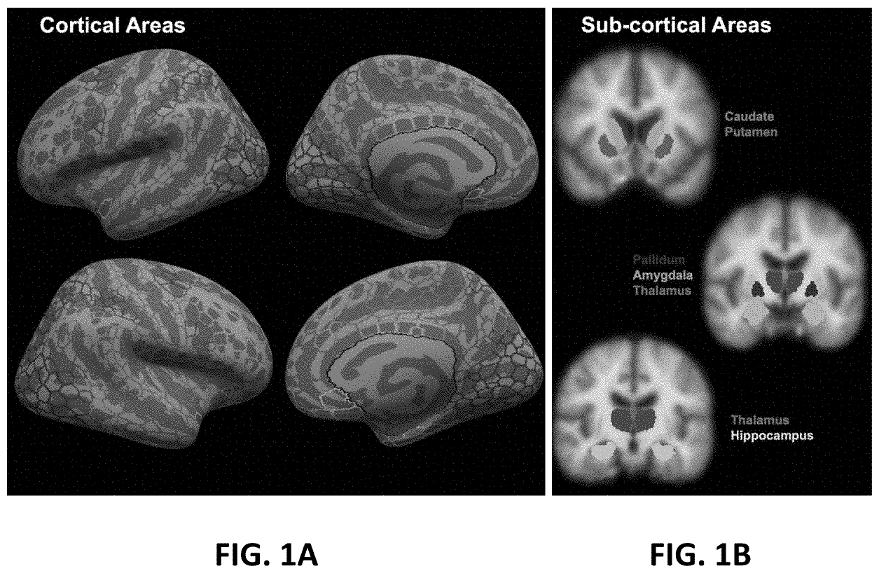

Image Preprocessing and data extraction. The recon-all pipeline from the FreeSurfer toolbox (Version 6.0; https://surfer.nmr.mgh.harvard.edu) was used to process the anatomical brain images. Schaefer's atlas was used to parcellate the whole brain into 1000 cortical (FIG. 1A) (https://pubmed.ncbi.nlm.nih.gov/28981612/) and 12 sub-cortical (FIG. 1B) regions. Subject-wise measures of CVol for cortical areas, SCV for sub-cortical areas, and intracranial volume (ICV; a measure of head size) were evaluated separately for the left and the right hemisphere using the mri_surf2surf, mris_anatomical_stats, and aparcstats2table pipelines following recon-all pipeline from FreeSurfer.

Data preparation. Estimation of CVol values of 1000-regions (i.e., 500 regions for each hemisphere) and SCV of 12 sub-cortical regions (i.e., 6 sub-cortical regions for each hemisphere) for each participant resulted in a total of 1012 features corresponding to volumetric measures (i.e., CVol and SCV) for each subject. Prior to performing feature selection and machine learning analysis, region-specific CVol/SCV were adjusted with respect to age, sex, IQ, and head size (i.e., ICV). This was performed in MATLAB R2022a by evaluating residualized volumetric parameters of each region.

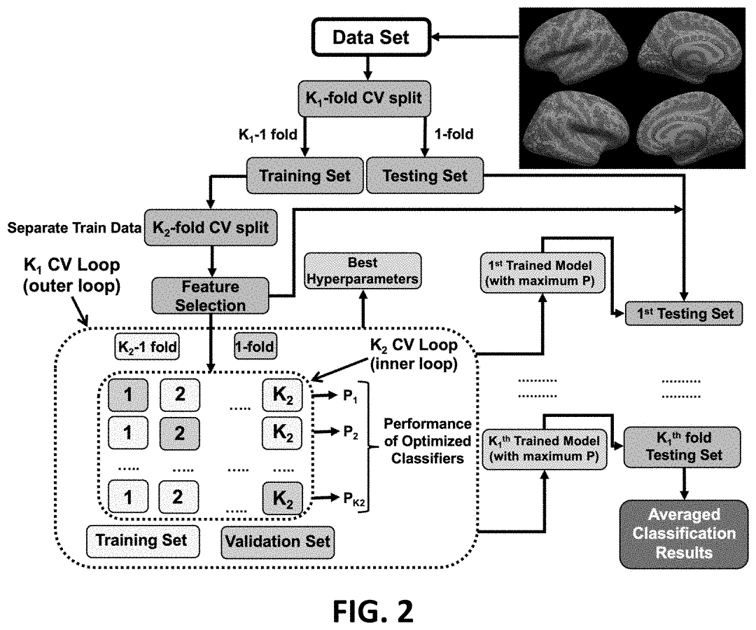

Feature Selection and Machine Learning Analysis. Feature selection and machine learning analysis were also performed in MATLAB R2022a. The example used K₁×K₂ (here, K₁=10 and K₂=10) nested cross-validation (also called two-step, two-layer cross-validation) approach where K₁ and K₂ represent the number of outer and inner loops respectively. The following steps (steps 1-10) were performed for CVol/SCV data set (see, e.g., FIG. 2 for the detailed illustration of the procedure):

Step 1: The data set was randomized prior to performing feature selection and machine learning analysis.

Step 2: In the outer loop, data was split into K1 folds where K1-1 folds were used as training data set and one-fold was used as a test data set.

Step 3: The training data set was transformed into z-score and the corresponding transformations were applied to test data set.

Step 4: In the inner loop, the training data set was divided into K2 folds to be used as a sub-training (K2-1 folds) and validation data sets (one-fold).

Step 5: The least absolute shrinkage and selection operator (LASSO) feature selection was applied on the sub-training data set. This procedure was then repeated K2 times, resulting in K2 sets of best features. The final set of features were comprised of features that appeared at-least 50% times in K2 sets of best features.

Step 6: The classifier was then trained using this sub-training data set (now revised with final set of features) and tested using the validation data set. Bayesian Optimization was used for parameter tuning during this step.

Step 7: The process was repeated K2 times which resulted in K2 number of validation accuracies. The model that gave the maximum validation accuracy with best optimized hyper parameters was then tested on the test data set in the outer loop, repeated K1 times.

Step 8: The accuracies, sensitivity parameters, specificity parameters, and AUC parameters corresponding to each test fold were averaged to find the generalized accuracy (ACC), generalized sensitivity (SEN), generalized specificity (SPEC), and generalized AUC of the model.

Step 9: The rocmetrics implemented in MATLAB R2022a was used to estimate area under the receiver operating characteristics curve (AUC).

In this example, three different classification algorithms were used to train the model, namely, the support vector machine (SVM), k-Nearest Neighbors (k-NN), and classification ensemble (ENS), and the ACC, SEN, SPEC, and AUC were estimated from each of these three classification algorithms.

The features that were bilaterally common across each of the K1 folds were finally identified and reported.

Results

Feature identification. A total of 62 features were identified through CVol/SCV parameters. This included the brain regions with lower CVol for adolescents at suicide risk (relative to typically developing adolescents) within the bilateral orbitofrontal cortex, bilateral inferior frontal gyrus, bilateral superior frontal gyrus, regions within the bilateral inferior/middle/superior temporal gyrus, and bilateral fusiform gyrus, and brain regions with greater CVol for adolescents at suicide risk (relative to typically developing adolescents) within the bilateral cuneus/precuneus.

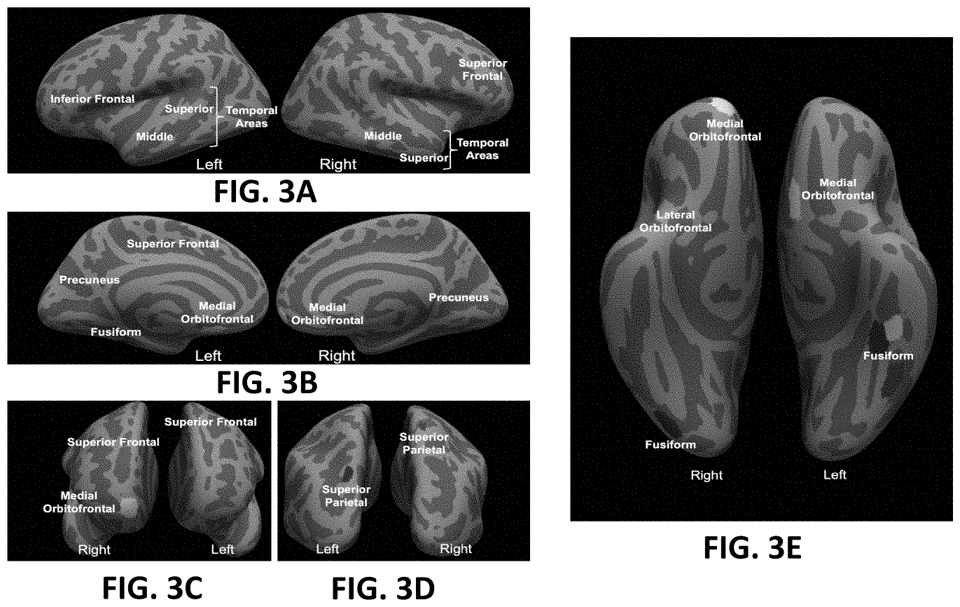

Model Performance. It was possible to predict whether an individual was at high or low suicide risk with high ACC, SEN, SPEC, and AUC of 74.79%, 75.90%, 74.07%, and 87.18% respectively; see, e.g., FIGS. 3A-3E for bilateral anatomical locations of identified sMRI biomarkers of suicide risk. Table 1 shows the ACC, SEN, SPEC, and AUC for CV/SCV of identified features and for each classification algorithms (i.e., SVM, k-NN, and ENS). SVM was the best performing classifier among all the three classification algorithms.

TABLE 1
Model Performance

| Morphometry | | ACC | SEN | SPEC | AUC |
|---|---|---|---|---|---|
| Parameters | Algorithms | (%) | (%) | (%) | (%) |
| CV/SCV | SVM | 74.79 | 75.90 | 74.07 | 87.18 |
| | k-NN | 73.12 | 78.72 | 68.03 | 81.42 |
| | ENS | 63.54 | 66.68 | 62.47 | 80.28 |

Summary

The current example suggests widespread structural brain alterations associated with suicide risk. The proposed approach (i.e., 1000-area whole-brain parcellation in conjunction with cortical/sub-cortical parameters and cutting-edge machine learning technique) is very efficient in determining the spatial extent of altered cortical/sub-cortical structures associated with suicide risk.

Therefore, it is shown that using the methods and systems disclosed herein can provide numerous advantages and potential benefits for doctors, teachers, therapists, nurses, care providers, and generally anyone associated with the detection, diagnosis, and/or treatment of a mental illness. The training model and application thereof will be invaluable for clinical centers working with potentially suicidal adolescents across the nation and, indeed, globally, as it has the potential to decrease the number of suicides and provide better treatment for people of all ages who may be experiencing mental issues. By tracing the issues to biomarkers of the brain, the resulting diagnosis and treatment can be treated in a more agnostic manner to provide the tools needed to combat mental issues. This may also give an indication as to the prognosis of some treatments.

Still further, has been included, the aspects and/or embodiments disclosed herein should not be limited to suicide or suicide risk. Indeed, it is to be appreciated that the training model could be trained to identify any number of biomarkers that may be linked to generally any mental and/or behavioral issue. The same method provided could be utilized to train the model to identify classifiers associated with said other mental and/or behavioral issues, and the model could then be applied in clinics, hospitals, or wherever to attempt to identify potential issues at an early as possible stage to provide the best basis for treatment of the same. As noted, such mental and/or behavioral issues can include, but should not be limited to, depression, aggression, anxiety, and/or conduct disorders, in addition to the suicide and suicide risk described herein. As noted, the training of the model will generally take the same steps, but the classifiers will be changed for the types of conditions being trained.

From the foregoing, it can be seen that the invention accomplishes at least all of the stated objectives.

1. A system for diagnosing and/or prognosing mental issues, comprising:
at least one processor and at least one memory configured to implement a deployed learning network model, the deployed network model generated from a training network, wherein the training network trained with a method comprising the steps of:
reviewing a plurality of brain images comprising a plurality of regions of the brain; and
identifying a classifier of the brain images that corresponds with one or more mental issues; and
the deployed learning network stored on one or more non-transitory computer readable media comprising instructions comprising:
comparing the deployed learning network model with brain images of a patient to determine if the brain images of the patient correspond to the one or more mental issues associated with the trained classifier.

2. The system of claim 1, wherein the network model comprises a nested cross-validation approach.

3. The system of claim 1, wherein the training network comprises:
a. a support vector machine (SVM) method,
b. a k-Nearest Neighbors (k-NN) method, or
c. a classification ensemble (ENS) method.

4. The system of claim 3, wherein the training network method further comprises fining generalized accuracy of the deployed network model.

5. The system of claim 1, wherein the brain images comprise whole-brain parcellation into 1000-cortical regions and 12-subcortical regions.

6. The system of claim 5, wherein the 1000-cortical regions are separated into 500 regions per hemisphere.

7. The system of claim 5, wherein the 12-subcortical regions are separated into 6 regions per hemisphere.

8. The system of claim 1, wherein the brain images are adjusted with respect to age, sex, IQ, and head size.

9. The system of claim 1, wherein the one or more mental issues comprises risk of suicide.

10. A computer implemented method, comprising:
training a network via a processor, wherein the training the network comprises:
reviewing a plurality of parcellated brain images;
identifying one or more classifiers of the parcellated brain images that corresponds to one or more mental conditions; and
storing the classifiers in a memory associated with the processor;
using the trained network to compare stored classifiers with brain images of a patient to diagnose one or more mental conditions of the patient.

11. The method of claim 10, further comprising determining a treatment for the diagnosed mental condition based upon the diagnosis with the trained network.

12. The method of claim 10, wherein the one or more mental conditions comprises suicide risk.

13. The method of claim 10, wherein the plurality of parcellated brain images comprise images separated into 1000-cortical regions and 12-subcortical regions.

14. The method of claim 10, wherein the plurality of parcellated brain images comprise structural MRI (sMRI) data.

15. The method of claim 10, wherein the one or more classifiers were determined using a support vector machine learning model.

16. The method of claim 10, wherein the one or more identified classifiers comprise biomarkers associated with brain regions.

17. A method for identifying mental issues, comprising:
training a network stored on a processor with memory by:
reviewing a plurality of brain images;
identifying one or more classifiers of the brain images that corresponds to a mental condition; and
storing the classifiers in the memory;
using the trained network to compare stored classifiers with brain images of a patient to identify potential of the mental condition of the patient.

18. The method of claim 17, wherein the plurality of brain images comprises parcellated brain images.

19. The method of claim 18, wherein the plurality of parcellated brain images comprise images separated into 1000-cortical regions and 12-subcortical regions.

20. The method of claim 17, wherein the training network comprises:
a. a support vector machine (SVM) method,
b. a k-Nearest Neighbors (k-NN) method, or
c. a classification ensemble (ENS) method.
